(19) United States
(12) Patent Application Publication
(10) Pub. No.: US 2025/0003991 A1
(43) Pub. Date: Jan. 2, 2025

(54) METHOD FOR MEASUREMENT OF HEMOPROTEIN IN A SAMPLE

(71) Applicants: Universitair Zeikenhuis Antwerpen, Edegem (BE); Universiteit Antwerpen, Antwerpen (BE)

(72) Inventors: Matthias CUYKX, Bercham (BE); Bart PEETERS, Antwerpen (BE); Herman GOOSSENS, Wilrijk (BE)

(21) Appl. No.: 18/690,572

(22) PCT Filed: Sep. 9, 2022
(86) PCT No.: PCT/EP2022/075055

§ 371 Date: Mar. 8, 2024

(30) Foreign Application Priority Data
Sep. 9, 2021 (EP) 21195789.9

(51) Int. Cl. G01N 33/72; G01N 21/31

(52) U.S. Cl. CPC G01N 33/721; G01N 21/31; G01N 2201/127

(57) ABSTRACT

Provided herein is a method for determining a concentration of a hemoprotein in a test sample, wherein the hemoprotein comprises heme prosthetic group containing porphyrin, the method comprising contacting the test sample with a modification composition comprising at least one modification reagent for degrading the porphyrin, measuring spectrophotometrically a change in an absorbance profile of the porphyrin caused by the degradation, wherein the absorbance profile is measured within a spectrophotometric range of between 510 nm and 590 nm, and determining from the change in the absorbance profile, the concentration of hemoprotein in the sample.

### FIELD OF THE INVENTION

The presented methods and kits are for the detection of hemoprotein in a test sample, more in particular, for detection of hemoglobin in a test sample.

### BACKGROUND TO THE INVENTION

Haemolysis in vivo can be caused by several factors, ranging from auto-immune haemolytic anemia, sickle cell anemia to drug-induced haemolysis. Venovenous and venoarterial extracorporeal membrane oxygenation (ECMO) is a method often applied to sustain blood oxygenation during cardiac surgery and severe COVID19 pneumonia. The associated haemolysis is a possible complication during therapy and needs to be carefully monitored.

Measurement of concentration of hemoglobin present in the plasma, i.e. hemoglobin not incorporated within a red blood cell, is used to objectify and monitor the severity of haemolysis. Free haemoglobin is a good indicator to monitor such haemolytic events, as it quickly rises when haemoglobin scavengers such as haptoglobin are consumed.

Available methods are labour intensive (not automated), are prone to interference, lack analytical sensitivity or use toxic reagents (e.g. benzidine derivates, Drabkin reagent). Currently there is no recommended method nor standardized kit available on the market with approval of competent authorities (e.g., In vitro Diagnostic label from United State Federal Drug Administration or CE (Conformité Européenne) label from European Community). None of the major leader in clinical biochemistry automation offers comprehensive assays to clinical laboratories.

A method typical of the art is described in, for example, Barr I. et al.: “Pyridine hemochromagen assay for determining the concentration of heme in purified protein solutions”, Bio Protoc., vol. 5, no. 18, E1594; the technique is a multi-step method that detects spectrophotometric changes caused by iron oxidation, and requires quantitative oxidation of the sample and its subsequent reduction. The method uses two reaction steps, and is time consuming and costly in terms of reagents, steps, measurements (wavelength scans), and reaction waiting times.

A commonly-used reagent is Drabkin's reagent, as used, for instance, in EP0158506, however, it is known to produce errors (Beal et al. “Drabkin's solution: A common source of error in haemoglobinometry”, Pathology, Volume 6, issue 3, p251-254, 1974), and is not considered reliable. In addition Drabkin's reagent contains toxic components, such as cyanide.

Methods which use a chromophore to detect progress of an redox reaction (e.g. WO 9735030) suffer from inaccuracies in a clinical setting. These methods use 3-4 steps incurring a high variability. Timings of the steps and volume/weight measurements need to be observed and consistent. Any variation has an effect per operator, that is amplified for multiple operators (e.g. day and night operators), leading to inaccuracies. Moreover, chromophore used is benzidine, which has a negative impact on the health of the user and on the environment.

Presented herein a method and kit for measurement of hemoprotein concentration that overcomes the problems of the art.

### SUMMARY OF THE INVENTION

Provided herein is a method for determining a concentration of a hemoprotein in a test sample comprising contacting the test sample with a modification composition comprising at least one modification reagent for changing a chemical and/or physical property of the hemoprotein, measuring spectrophotometrically a change in an absorbance profile of the heme caused by the modification, and determining from the change in the absorbance profile, the concentration of hemoprotein in the sample.

More in particular, provided herein is method for determining a concentration of a hemoprotein in a test sample, wherein the hemoprotein comprises heme prosthetic group containing porphyrin, the method comprising:
contacting the test sample with a modification composition comprising at least one modification reagent for degrading the porphyrin,
measuring spectrophotometrically a change in an absorbance profile of the porphyrin caused by the degradation, wherein the absorbance profile is measured:
   at one wavelength within a spectrophotometric range of between 510 nm and 590 nm, or
   at a plurality of wavelengths at least one of which is within a spectrophotometric range of between 510 nm and 590 nm, and
      determining from the change in the absorbance profile, the concentration of hemoprotein in the sample.

The modification reagent is preferably present in an amount to cause a loss of aromaticity to the porphyrin in the test sample.

The at least one modification reagent may be an oxidation reagent, an acid, or a radical agent.

The at least one modification reagent may be H₂O₂ (1-5% v/v), HNO₃ (2N), HClO₄ (0.5-2% v/v), metavanadate (1-6% mmol/L), HCl (1-2N), or NaOCl (1-5% v/v).

Preferably, the at least one modification reagent is H₂O₂, HNO₃, HClO₄, metavanadate, HCl, NaOCl, sodium nitrate, tetrachloro-methane, bromo-trichloromethane, cumene hydroperoxide (CHP), t-butyl hydroperoxide, t-butyl perbenzoate, and dibenzoyl peroxide, nitrates, ozone, or a peroxide.

The absorbance profile may be measured at one wavelength within a spectrophotometric range of between 510 nm and 590 nm, or at a plurality of wavelengths at least one or more of which is within a spectrophotometric range of between 510 nm and 590 nm.

The change in absorbance profile caused by the modification composition may be determined by:
measuring a first absorbance profile (AT1) of the test sample prior to contact with the modification composition;
measuring a second absorbance profile (AT2) of the test sample after contact with the modification composition and after the hemoprotein has been modified;
calculating a difference between the first (AT1) and second (AT2) absorbance profiles.

The step of determining from the change in absorbance profile the concentration of hemoprotein in the test sample may comprise applying a calibration constant (Ct) to the change in absorbance profile.

The concentration (CTH) of hemoprotein in the test sample may be determined by the equation:

$$CTH = {( 1 / ( Ct ) )}^{*} ( AT 1 - AT 2 )$$

wherein AT2 has been corrected for any change in volume caused by contacting the test sample with the modification composition.

The calibration constant (Ct) may be determined by measuring spectrophotometrically a change in absorbance profile caused by modification of a known concentration (CCH) of hemoprotein in a calibration sample.

The change in absorbance profile caused by modification of the known concentration (CCH) hemoprotein in the calibration sample may be determined by:
measuring a first absorbance profile (AC1) of the calibration sample prior to addition of the modification composition;
measuring a second absorbance profile (AC2) of the calibration sample after addition of the modification composition and after the hemoprotein has been modified; and
calculating a difference between the first absorbance profile (AC1) and the second absorbance profile (AC2), and
   the calibration constant (Ct) is calculated by the equation:
   Ct=1/(CCH)*(AC1−AC2) wherein AC2 has been corrected for any change in volume caused by contacting the calibration sample with the modification composition.

The concentration (CTH) of hemoprotein in the test sample may be determined by the equation:

$$CTH = 1 / {Ct}^{*} ( AT 1 - AT 2 )$$

wherein AT2 has been corrected for any change in volume caused by contacting the test sample with the modification composition.

The change in an absorbance profile of the porphyrin caused by the degradation, preferably comprises:
a reduction in absorbance at one wavelength within a spectrophotometric range of between 510 nm and 590 nm, or
a reduction in absorbance at a plurality of wavelengths at least one of which is within a spectrophotometric range of between 510 nm and 590 nm.

Preferably, the hemoprotein is hemoglobin.

The hemoprotein in the test sample and in the calibration sample may be the same type, preferably hemoglobin.

The hemoprotein in the test sample may be present in blood plasma, urine or cerebrospinal fluid.

Further provided is a kit for determining a concentration of hemoprotein in a test sample, comprising a means for spectrophotometrically measuring modification of the hemoprotein in the sample, which means comprises a modification composition.

Preferably, the means comprises
a stock solution of 30% (v/v) hydrogen peroxide,
a diluent for diluting the stock solution to a concentration of 1% to 5% (v/v).

The kit may further comprise:
a stock calibration standard for dilution containing haemoprotein at known concentration, and/or
a plurality of calibration standards, each containing hemoprotein at a different known concentration.

Further provided is a computer-implemented method for determining a concentration of a hemoprotein in a test sample as described herein.

Further provided is a computer-implemented method for determining a concentration of a hemoprotein in a test sample comprising
receiving data of the spectrophotometrically measurements as described herein, and
determining the concentration of hemoprotein as described herein.

Further provided is a computer-implemented method for determining a concentration of a hemoprotein in a test sample comprising:
receiving data comprising a first absorbance profile of the test sample prior to contact with the modification composition;
receiving data comprising a second absorbance profile of the test sample after contact with the modification composition and after the hemoprotein has been modified;
determining from the change in the absorbance profile, the concentration of hemoprotein in the sample.

### FIGURE LEGENDS

FIG. 1 shows a graph of hemoglobin concentration in a plasma sample measured according to the present method and the benzidine method. Line (a) represents the upper confidence interval of the regression line, line (b) is the best data fit, line (c) is an ideal fit, line (d) is the lower confidence interval of the regression line.

FIG. 2 shows a graph of hemoglobin concentration in a plasma sample measured according to the present method and a direct spectrophotometry using Roche Cobas 8000 spectrophotometer.

FIG. 3 shows a graph of recovery of hemoglobin concentration in a plasma sample measured according to the present method, wherein the sample is contaminated by increasing amounts of bilirubin.

FIG. 4 shows a graph of recovery of hemoglobin concentration in a plasma sample measured according to the present method, wherein the sample is contaminated by increasing amounts of lipid particles.

FIG. 5A to 5H, photometric spectra of hemoglobin ((A), no modification reagent) contacted with different modification compositions each containing a different candidate modification reagent H₂O₂ (3% v/v) (B), HNO₃ (2N) (C), HClO₄ (1% v/v) (D), Metavanadate (4 mmol/L) (E), HCl (2N) (F), NaOCl (5% v/v) (G), Filtration (H).

FIG. 6 bar graph showing distribution of free hemoglobin concentrations in clinical patients as measured by the present method.

FIG. 7 Plot showing evolution over time of free hemoglobin concentration in a patient undergoing extracorporeal membrane oxygenation (ECMO) as measured by the present method. Arrow indicates an intervention after detecting a high level or hemolysis.

### DETAILED DESCRIPTION OF INVENTION

Before the present methods and kits of the invention are described, it is to be understood that this invention is not limited to particular methods and kits or combinations described, since such methods and kits and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, >4, ≥5, ≥6 or >7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense, and the scope of the present invention is defined by the appended claims.

Provided herein are methods and kits for determining a concentration of hemoprotein in a test sample by spectrophotometrically measuring a change (e.g. intensity decrease, peak shift) in an absorbance profile of the heme present, in particular of the porphyrin present, in the hemoprotein caused by a physical and/or chemical modification of the hemoprotein, in particular of the porphyrin. The inventors have found that the modification causes a change in the spectrophometric profile i.e. at one or more wavelengths within heme porphyrin absorbance spectrum (510 nm to 590 nm), and that the change is indicative of the concentration of hemoprotein in the test sample.

Provided is a method for determining a concentration of hemoprotein in a test sample comprising contacting the test sample with a modification composition comprising at least one modification reagent for changing a chemical and/or physical property of the hemoprotein, measuring spectrophotometrically a change in an absorbance profile of the heme, in particular of the porphyrin, caused by the modification, and determining from the change in the absorbance profile, the concentration of hemoprotein in the sample.

Adding a single composition to the sample simplifies processing of multiple samples. In robotic processing using colour change reagents, multiple stock reagent bottles are used, and there is a pre-mixing of reagents prior to dispensing which is a source of inaccuracy for small volume samples. Colour reagents often comprise of complex aromatic structures, and are often toxic to human or animal health.

The reaction of the present invention does not need to be stopped. The reaction mixture can be left indefinitely, all factors being equal (e.g. no evaporation). In a clinical setting, this requires less steps (no quenching), saving time and costs. Because of the less steps, there is more pipetting and reaction time accuracy per operator, and a reduction in inter-operator variability. The time from treating the sample to measuring the absorbance is non-critical. Hence, these advantages lead to a superior accuracy compared to other methods of the art.

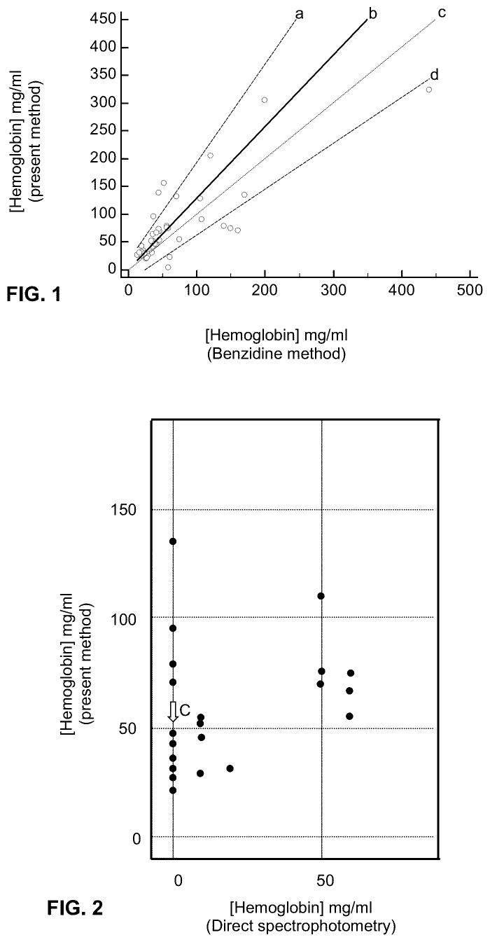

The method avoids the use of chromophores, such as benzidine which is now obsolete due to its carcinogenic potential. Nonetheless, measurements using the present method correlate well with the benzidine method (Example 2 and FIG. 1)

The method is more reliable than other direct spectrophotometric measurements. For instance, the method employed by the Roche Cobas 8000 spectrophotometer does not yield precise results at lower, physiological relevant concentrations (see Example 3 and FIG. 2)

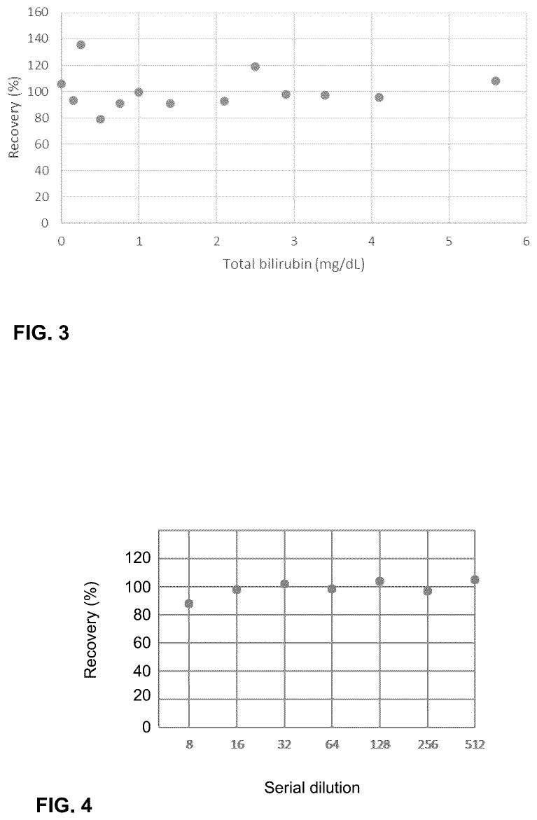

The method demonstrates little or no interference from bilirubin e.g. in subjects suffering from icterus (jaundice)—see Example 4 and FIG. 3)

The method demonstrates little or no interference from lipaemia e.g. in subjects suffering from an abnormally high concentration of emulsified fat in the blood—see Example 5 and FIG. 4.

The method is reliable in physiological concentration ranges. Accuracy and precision strongly outperform the accuracy of the manual methods. The accuracy and precision of the method around the clinical level of 40 mg/L is significantly better than the precision described in direct spectrophotometric assays (see Example 3). The method has a significantly lower limit of detection, being the only method to reliably measure hemoprotein at levels below the clinically relevant concentration of 40 mg/L; thus a sample from a subject plasma can be measured without additional preparation steps such as dilution.

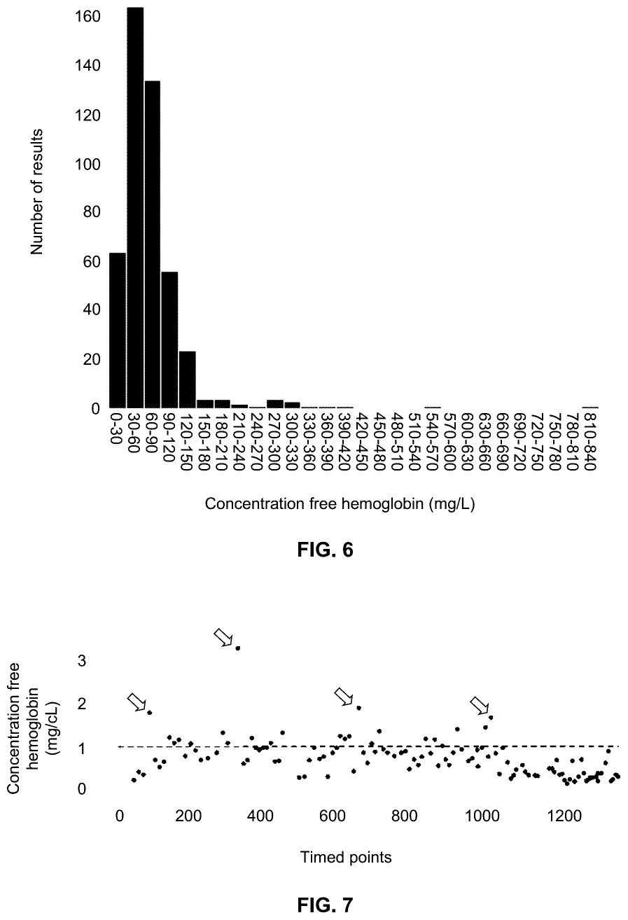

The present method has a wide dynamic range; it has been validated for measurement of concentrations up to around 1000 mg/ml (Example 7 and FIG. 6). Hence, the sample does not need to be diluted for patient samples in a clinical setting, even for outliers well outside the normal range (e.g. FIG. 6, 830 mg/L).

The present method has a proven utility and accuracy in a clinical setting (Example 8 and FIG. 7), in, particular for monitoring free hemoglobin in a patient undergoing extracorporeal membrane oxygenation (ECMO).

The method is applicable to any hemoprotein because all hemoproteins contain porphyrin. Because the method monitors degradation of porphyrin, the peroxidase unreactivity/reactivity is irrelevant. Hence, it is not reliant on redox chromophores.

The test sample is a sample whose concentration of hemoprotein is to be determined. The test sample may be liquid. The test sample may be mammalian, human, or animal. The test sample preferably contains hemoprotein of only one type (e.g. contains hemoglobin with undetectable or no myoglobin, or contains myoglobin and undetectable or no hemoglobin). The test sample may be blood plasma (typically containing hemoglobin and undetectable or no myoglobin). In blood plasma, the hemoglobin, sometimes called free hemoglobin, is not contained in red blood cells. The test sample may be urine or cerebrospinal fluid.

The concentration range of hemoprotein in the test sample may be 30 to 150000 nmol/L. Where the hemoprotein is hemoglobin, the concentration range in the test sample may be 2 mg/L to 10 000 mg/L; the useful range for detection of haemolysis 40 mg/L or less. The limits of the concentration may be determined according to the absorbance detection limits of the spectrophotometer.

The calibration sample is a sample whose concentration of hemoprotein is known. The calibration sample may be liquid. The calibration sample may be mammalian, human, or animal. The hemoprotein may be extracted from a biological sample, or may be over-expressed in vitro from a gene. The calibration sample preferably contains hemoprotein of only one type (e.g. hemoglobin with undetectable or no myoglobin, or myoglobin and undetectable or no hemoglobin). The calibration sample may be blood plasma.

The number of calibration samples may be one or a plurality. The plurality of calibration samples may contain at least two (e.g. 1, 2, 3, 4, 5, or more) calibration samples containing different concentrations of hemoprotein. The concentration of the one or more calibration samples is typically chosen to be within absorbance detection limits of the spectrophotometer. The concentration of the one or more calibration samples is typically chosen to be within the range of the test sample. The concentration range of hemoprotein in a calibration sample may be 200 to 15500 nmol/L, for instance 200 to 1000 nmol/L. Where in the hemoprotein is hemoglobin, concentration range in the calibration sample may be 15 mg/L up to 1000 mg/L. Where a plurality of calibration samples is provided, one may be at a concentration within a range of 20-50 mg/L hemoglobin, and another may be at a concentration within a range of 100-500 mg/L hemoglobin. In a clinical setting for detection of hemolysis, as a guidance, one calibration sample may contain 50 mg/L hemoglobin and another calibration sample may contain 350 mg/L hemoglobin.

The concentration of the hemoprotein in the calibration sample may be verified by independent means, such as using a hematology analyser. A hematology analyser such as a flow cytometer (e.g. Sysmex 9000 hematology analyser) is typically used to measure hemoglobin levels in whole blood samples; for determining concentrations of hemoprotein concentration in the calibration sample, it is used to first measure accurately concentration of a concentrated stock solution (e.g. 3 g/dL) which is within its measurement range. The concentrated stock solution is subsequently diluted within a range of the calibration sample.

The type of hemoprotein in the calibration sample and in the test sample is preferably the same. For instance, where a concentration of hemoglobin is to be measured in the test sample, the calibration sample also contains hemoglobin at a known concentration.

Hemoprotein refers to any complex containing heme non-covalently bound to at least one polypeptide chain. It can be prepared from a biological sample (blood, plasma, urine, cerebrospinal fluid), but may be recombinant. The hemoprotein comprises polypeptide and heme prosthetic group containing porphyrin. The porphyrin is typically associated with iron in clinical subjects.

Principal examples of a hemoprotein are hemoglobin and myoglobin. Other examples of a hemoprotein include neuroglobin, cytoglobin, and leghemoglobin. Further examples include enzymes such as cytochrome P450s, cytochrome c oxidase, ligninases, catalase and peroxidase. Further examples include cyctochrome a, cytochrome b, and cytochrome c have such electron transfer functions.

The hemoprotein contains at least one heme. The type of heme may be any, such as, for instance, the most common types heme A, heme B, heme C, heme O. Other types of heme include, heme I, heme m, heme D, Heme S.

The hemoprotein contains at least one porphyrin (i.e. at least one porphyrin ring). The hemoprotein typically contains between 1 and 4 porphyrins. Hemoglobin contains 4 porphyrins. Myoglobin contains 1 porphyrin.

The method is applicable to any hemoprotein because all hemoproteins contain porphyrin. Because the method monitors degradation of porphyrin, the peroxidase unreactivity/reactivity is irrelevant.

The hemoprotein detected is preferably hemoglobin, more preferably free hemoglobin. The hemoprotein is preferably in a plasma sample.

Each absorbance profile (before contact with the modification composition, after contact with the modification composition, in the test sample or in the calibration sample) contains spectrophotometric measurement data of the sample. The absorbance profile may contain any type of spectrophotometric measurement data, for instance, one or more of single wavelength measurement data, multiple discrete wavelength measurement data, wavelength scan measurement data, measurement data acquired at a single time point (end-point), measurement data acquired at a multiple time points (kinetic), spectrophotometric measurement data that has been processed (e.g. statistically processed (average, median), integrated)). The spectrophotometric measurement is made at least within the porphyrin absorbance spectrum range (e.g. between 510 nm and 590 nm, or preferably 570 nm to 580 nm, or more preferably 571 nm to 577 nm). The spectrophotometric measurement is preferably made only within the porphyrin absorbance spectrum range.

The spectrophotometric measurement may be made at least within the heme absorbance spectrum range (e.g. between 510 nm and 590 nm, or preferably 570 nm to 580 nm, or more preferably 571 nm to 577 nm). The spectrophotometric measurement may preferably be made only within the heme absorbance spectrum range.

Each and every absorbance profile (before contact with the modification composition, after contact with the modification composition, in the test sample or in the calibration sample) is preferably recorded at the same wavelength or wavelengths or within a tolerance of ±10 nm.

The absorbance profiles before and after contact with the modification composition are preferably measured at the same wavelength or wavelengths or within a tolerance of ±10 nm. The absorbance profiles of the test sample and calibration sample are preferably measured at the same wavelength or wavelengths or within a tolerance of ±10 nm.

Each and every absorbance profile (before contact with the modification composition, after contact with the modification composition, in the test sample or in the calibration sample) is preferably recorded using the same spectrophotometer. The absorbance profile of the test sample and of the calibration sample are preferably recorded using the same spectrophotometer. The absorbance profile of the test sample before and after contact with the modification composition are preferably recorded using the same spectrophotometer. The absorbance profile of the calibration sample before and after contact with the modification composition are preferably recorded using the same spectrophotometer.

The change in absorbance profile may concern a change in absorbance value at one or more given wavelengths (e.g. 571 nm or 574 nm). The change in absorbance profile may concern a reduction in absorbance value at one or more given wavelengths (e.g. 571 nm or 574 nm). The change in absorbance profile may concern a change (shift) in peak wavelength (e.g. a shift from 571 nm to 550 nm).

The absorbance profile after contact with the modification composition (second absorbance profile, AT2, or AC2) may be acquired at an end-point at which the modification of all the hemoprotein in the test sample or calibration sample is complete. The end point is typically reached within 5, 10 or 15 minutes after contact with the modification composition. The end end-point may be determined by monitoring spectrophotometric absorbance of the test sample or calibration sample over time until there is no trend of change.

The absorbance profile after contact with the modification composition (second absorbance profile, AT2, or AC2) may be derived from several spectrophotometric measurements recorded at different time points. The absorbance profile may hence contain reaction kinetic data such as reaction speed.

Each and every absorbance profile (before contact, after contact, test sample and calibration sample) may comprise an (one) absorbance value measured at a single discrete wavelength. The single discrete wavelength may be a value in a range between 510 nm and 590 nm, or preferably 570 nm to 580 nm, or more preferably 571 nm to 577 nm. The single discrete wavelength may have the same value in each and every absorbance profile (before contact, after contact, test sample and calibration sample), or vary by no more than +10 nm.

Each and every absorbance profile (before contact, after contact, test sample and calibration sample) may comprise absorbance values measured at multiple different wavelengths. The multiple different wavelengths may be discrete absorbance values, or sequential values as part of a continuous range (wavelength scan). One, one or more, a majority, or all of the multiple different wavelengths may be within a range between 510 nm and 590 nm, or preferably 570 nm to 580 nm, or more preferably 571 nm to 577 nm. The range may span 80 nm, preferably 10 nm, more preferably 6 nm. The multiple different wavelengths may be the same in each and every absorbance profile (before contact, after contact, test sample or calibration sample), or vary by no more than ±10 nm.

The multiple different wavelengths may be processed to give a scalar value, such as, for instance, obtaining an average absorbance, a median absorbance, or an integrated absorbance. One example of averaging is measurement of absorbances at 500 nm, 571 nm and 600 nm, and the absorbance profile is the absorbance at 571 nm minus the average between 500 and 600 nm.

One or more additional processing steps may be included in the absorbance profile measurement (before contact, after contact, test sample and calibration sample) in order to further improve accuracy. For instance, the absorbance profile may be subtracted from a baseline. The baseline may be estimated from several absorbance points (e.g. a wavelength scan between 300 and 600 nm, or absorbance points 300, 450, 500 and 600 nm). Typically, a spectrophotometer will automatically perform a baseline calibration across its working wavelength range, using air or water as the baseline.

The modification composition comprises one or more modification reagents. The modification composition may be a liquid or solid. The modification composition may contain only one modification reagent. The modification composition may contain several modification reagent.

The modification reagent changes one or more properties of the hemoprotein, leading to a change in the absorbance profile of the heme. In particular, the modification reagent changes one or more properties of the porphyrin, leading to a change in the absorbance profile of the heme.

The modification reagent may change a chemical and/or physical property of the hemoprotein, leading to a change in the absorbance profile of the heme. The chemical and/or physical property of the hemoprotein may result in a chemical degradation of the hemoprotein, and/or in a precipitation of the hemoprotein, and/or affect the heme iron (e.g. conversion of ferro-iron to ferri-iron). The modification reagent is preferably for degrading the hemoprotein. The modification reagent may be a liquid or solid.

The modification reagent may change a chemical property of the polypeptide and/or heme. By chemical property, it is meant that the chemical structure of the polypeptide and/or heme is changed. The change to the chemical structure may involve one or more of: breaking of a covalent or non-covalent bond, modification of a chemical group, addition of a chemical group. A modification reagent that changes a chemical property of the polypeptide and/or heme may be an oxidation reagent. Examples of oxidation reagents include hydrogen peroxide, metavanadate and NaOCl.

The modification reagent may change a chemical property of the porphyrin. By chemical property, it is meant that the chemical structure of the porphyrin is changed. The change to the chemical structure may involve one or more of: loss of aromaticity, breaking of one or more covalent bonds, breaking one or more C═C bonds, most preferably a loss of aromaticity. A modification reagent that changes a chemical property of the porphyrin may be an oxidation reagent, and acid or a radical agent. Examples of oxidation reagents include hydrogen peroxide, metavanadate and NaOCl. Examples of acids include hydrochloric acid HCl, nitric acid HNO₃. Examples of radical agents include nitrates, ozone, hydrogen and organic peroxides, and halogenated radical agents.

The modification reagent preferably causes a (chemical) degradation of the porphyrin. The degradation of the porphyrin can be monitored by absorbance at 510 nm and 590 nm, preferably 570 nm to 580 nm, more preferably 571 nm to 577; during degradation, there is a decrease in absorbance. The degradation of porphyrin involves loss of aromaticity, breaking of one or more covalent bonds, breaking one or more C═C bonds, most preferably a loss of aromaticity. The loss of aromaticity can be detected spectrophotometrically from the porphyrin absorbance spectrum (i.e. decrease in absorbance between 510 nm and 590 nm, or preferably 570 nm to 580 nm, or more preferably 571 nm to 577 nm). A modification reagent that causes degradation of the porphyrin may be an oxidation reagent, an acid or a radical agent. Examples of oxidation reagents include hydrogen peroxide, metavanadate and NaOCl. Examples of acids include hydrochloric acid HCl, nitric acid HNO₃. Examples of radical agents nitrates, ozone, hydrogen and organic peroxides, and halogenated radical agents. Examples are given below.

The modification reagent may be an oxidation reagent. Examples of oxidation reagents include hydrogen peroxide H₂O₂, sodium hypochlorite NaOCl, metavanadate (e.g. ammonium metavanadate), sodium perchlorate HClO₄. The modification composition may comprise one or more of hydrogen peroxide H₂O₂ (e.g. 1-5% (v/v)), sodium hypochlorite NaOCl (e.g. 1-5% (v/v)), metavanadate (e.g. ammonium metavanadate e.g. 1-6 mmol/L), sodium perchlorate HClO₄ (e.g. 0.5-2% (v/v)).

The modification reagent may be a radical agent. Examples of radical agents include nitrates (e.g. nitric acid, sodium nitrate), ozone, halogenated radical agents (e.g. NaOCl, tetrachloro-methane, bromo-trichloromethane), peroxides (both hydrogen and organic peroxides), Examples of peroxides include hydrogen peroxide, cumene hydroperoxide (CHP), t-butyl hydroperoxide, t-butyl perbenzoate, and dibenzoyl peroxide. The modification composition may comprise one or more radical agents.

The modification reagent may be an acid. Examples of acids include hydrochloric acid HCl, nitric acid HNO₃. The modification composition may comprise one or more of HCl (e.g. 1-2N), nitric acid HNO₃ (e.g. 1-2N).

The modification reagent may be a precipitation reagent.

A candidate modification reagent may be routinely screened for suitability in the present method. Multiple calibration samples containing different concentrations of hemoprotein are contacted with the candidate modification reagent and absorbance profiles are measured before contact with the modification composition comprising the candidate modification reagent and after the modification has reached completion (end point). If there are differences in the before/after absorbance profiles that are proportional to the differences in concentrations of the hemoprotein, then the candidate modification reagent is suitable as a modification reagent in the present method. Candidate modification reagents were screened in Example 6.

The modification reagent is typically added in an equimolar amount or in stoichiometric excess (by moles) to the porphyrin in the sample. For instance, with a concentration of porphyrin containing proteins in the sample of 150 000 nmoles/L, the modification reagent is typically added to at least 5 times excess (by moles) of the maximal concentration of porphyrin. As a guidance, for a test or calibration sample volume of 1 ml, a 400-600 μl aliquot of modification composition may be added, wherein the modification composition contains a modification reagent that is H₂O₂ (3% v/v in the modification composition), HNO₃ (2N in the modification composition), HClO₄ (1% v/v in the modification composition), Metavanadate (4 mmol/L in the modification composition), HCl (2N), or NaOCl (5% v/v in the modification composition).

The modification composition is preferably the same in the test and calibration sample.

The modification composition may contain hydrogen peroxide. The modification composition contain hydrogen peroxide at a concentration of 1% to 5% (v/v), preferably 2% to 3% (v/v), more preferably 3% (v/v). One or more additives (e.g. anti-foaming agent) may or may not be added to improve reaction efficiency and reagent stability.

Where the modification reagent is hydrogen peroxide H₂O₂, and the concentration of H₂O₂ in the reaction mixture (i.e. after addition to the sample) is preferably 0.1 to 1% (v/v), more preferably 0.5 to 0.7% (v/v).

The modification reagent is preferably added in an amount to cause degradation (e.g. loss aromaticity) of the porphyrin in the sample. The degradation causes a reduction in the absorbance in the spectrophotometric range of between 510 nm and 590 nm. The reduction in the absorbance at the one wavelength or in the at least one of the wavelength of the plurality in the absorbance profile of the porphyrin caused by the degradation is proportional to the concentration of hemoprotein in the sample.

The porphyrin in the sample is degraded by the modification reagent. All the porphyrin in the sample is degraded by the modification reagent. By all the porphyrin in the sample is degraded by the modification reagent, it is meant that degradation (over time) is no longer detectable spectrophotometrically. Degradation of all porphyrin in the sample can be determined spectrophotometrically, where there is no longer a change (greater than noise) in absorbance between 510 nm and 590 nm, or preferably 570 nm to 580 nm, or more preferably 571 nm to 577 nm. The inventors have observed after around 5 minutes, flat line over time for a clinical sample containing a concentration of hemoglobin between 3 to 300 mg/L and with the addition of hydrogen peroxide solution to a concentration of ˜0.1% v/v.

The modification reagent is preferably present in an amount to cause chemical degradation (e.g. loss aromaticity) of the porphyrin in the sample within a time period. The time period may be less than 60 minutes, preferably less than 30 minutes, more preferably less than 30 minutes. The time period may be less than 1 to 10 minutes, preferably less than 1 to 5 mins. The amount can be determined spectrophotometrically, for instance, by adjusting the amount of modification reagent present until there is no longer a change (greater than noise) in absorbance between 510 nm and 590 nm, or preferably 570 nm to 580 nm, or more preferably 571 nm to 577 nm within the time period.

An accurate concentration of modification reagent is not critical because the reaction is a chemical degradation. The chemical degradation will proceed to completion over time. The reaction times are typically very short (less than 10 or 5 or 1 minute). Hence, there are no stoichiometric requirements, no need for accurate dispensing, and the reactivity of the modification reagent is less important. A larger amount of modification reagent will lead to a shorter reaction time.

The modification composition may further comprise one or more additives to improve reaction efficiency and reagent stability. An additive may be an anti-foaming agent. It is understood that one or more physical processes (e.g. heating, ultrasound) could be applied alone or additionally to the test or calibration sample to cause a change in a chemical and/or physical property of the hemoprotein.

The present method may not use a colour-change reagent. A colour change reagent is typically a redox chromophore. A redox chromophore is a substance that changes colour depending on its reductive-oxidative state. Examples of redox chromophores include 3,3′,5,5′-tetramethyl benzidine.

The present method may not use a wavelength outside the range 510 nm and 590 nm, or preferably 570 nm to 580 nm, or more preferably 571 nm to 577 nm.

The method may be a single reaction vessel method. In particular the steps of contacting and measurement of absorbance profiles are preferably performed in a single reaction vessel. Preferably the reaction vessel has one or more optically transparent windows. By optically transparent, it is meant that it allows passage of light for measurement of absorbance within the absorbance wavelength range of heme. The reaction vessel may be a cuvette, typically having at least 2 opposing sides that are optically transparent. The reaction vessel may be a well of a multiwall plate, typically having one side (a base) that is optically transparent, and an open lid. The material forming the optically transparent window may be transparent plastic (e.g. Poly(methyl methacrylate) (PMMA), polystyrene), glass or quartz.

After addition of the modification composition to the sample, the reaction is left to proceed to reach its end-point, which can be defined as a loss of aromaticity of porphyrin, as measured by a decline in absorbance between 510 nm and 590 nm, or preferably 570 nm to 580 nm, or more preferably 571 nm to 577 nm to a level indistinguishable from noise.

The modification reagent is preferably present in an amount to cause degradation of the porphyrin in the sample within a time period. The time period may be less than 60 minutes, preferably less than 30 minutes, more preferably less than 30 minutes. The time period most preferably may be less than 1 to 10 minutes, for instance, less than 1 to 5 mins.

Completion typically takes around 5 minutes in a clinical setting (e.g. for a clinical sample containing a concentration of hemoglobin between 3 to 300 mg/L and with the addition of hydrogen peroxide solution to a concentration of ˜0.1% v/v).

Advantageously, the reaction does not need to be stopped. The reaction mixture can be left indefinitely. In a clinical setting, this requires less steps (no quenching), and the time from treating the sample to measuring the absorbance is non-critical.

The concentration of hemoglobin in the test or calibration sample is determined from a change in the absorbance profile before and after contact with the modification composition. More in particular, it is determined from a difference in absorbance profiles before and after modification of the hemoprotein, in particular of the porphyrin. The difference may be corrected for any change in volume caused by contacting the test sample with the modification composition.

The change in absorbance profile caused by the modification in the test sample may be determined by:
measuring a first absorbance profile (AT1) of the test sample prior to contact with the modification composition;
measuring a second absorbance profile (AT2) of the test sample after contact with the modification composition and after the hemoprotein has been modified;
calculating a difference between AT1 and AT2.

Prior to calculating the difference, the second absorbance profile (AT2) may have been corrected as necessary for an effect of dilution caused by addition of the modification composition to the test sample. The correction may account for a change in volume caused by addition of the modification composition. Where the modification composition is a solid and there is no or negligible change in volume, correction might not be necessary.

The change in volume may cause a dilution. Where there is a dilution, the value of AT2 may be calculated by the formula:

AT2=AT2′((VTb)/(VTa))

where VTa is the test sample volume prior to addition of the modification composition, Where VTb is the test sample volume after addition of the modification composition, AT2′ is the uncorrected absorbance value measured after addition of the modification composition. The optical path length is constant when the absorbance is measured in the same cuvette.

Depending on the orientation of the light source and detector, the change in volume may further cause a change in optical path length. Where there is a path length change, the value of AT2 may be calculated by the formula:

$$AT 2 = AT 2 ’ ( ( VTb ) / ( VTa ) ) * ( ( P Ta ) / ( PTb ) )$$

where VTa is the test sample volume prior to addition of the modification composition, VTb is the test sample volume after addition of the modification composition, PTa is the test sample optical path length prior to addition of the modification composition, PTb is the test sample optical path length after addition of the modification composition, and AT2′ is the uncorrected absorbance value measured after addition of the modification composition. The optical path length may vary before and after measurement when the absorbance is measured in a multi-well plate.

The step of determining from the change in absorbance profile the concentration of hemoprotein in the test sample may comprise applying a calibration constant (Ct) to the change in absorbance profile.

The calibration constant (Ct) normalises the sample absorbance profile to an absolute hemoprotein concentration using the calibration absorbance profile of a known concentration of hemoprotein in the calibration sample.

The calibration constant (Ct) preferably scales the change in absorbance profile of the test sample. The concentration (CTH) of hemoprotein in the test sample may be determined by the equation:

$$CTH = {( 1 / ( Ct ) )}^{*} ( AT 1 - AT 2 )$$

The units of concentration of hemoprotein (CTH) is preferably mg/L and the units of the calibration constant (Ct) is preferably L/(mg*Absorbance units). It is with the scope of the present method that other concentration units are used.

The calibration constant (Ct) is determined by measuring spectrophotometrically a change in absorbance profile caused by modification of a known concentration (CCH) of hemoprotein in a calibration sample.

The change in absorbance profile caused by modification of the known concentration (CCH) hemoprotein in the calibration sample may be determined by:
measuring a first absorbance profile (AC1) of the calibration sample prior to addition of the modification composition; and
measuring a second absorbance profile (AC2) of the calibration sample after addition of the modification composition and after the hemoprotein has been modified;
calculating a difference between AC1 and AC2.

Prior to calculating the difference, second absorbance profile (AC2) may be corrected as necessary for an effect of dilution caused by addition of the modification composition to the calibration sample. The correction may account for a change in volume caused by addition of the modification composition. Where the modification composition is a solid and there is no or negligible change in volume, correction might not be necessary.

The change in volume may cause a dilution. Where there is a dilution, the value of AC2 is calculated by the formula:

$$A C 2 = A C 2 ’ ( ( VCb ) / ( VCa ) )$$

where VCa is the calibration sample volume prior to addition of the modification composition, VCb is the calibration sample volume after addition of the modification composition, AC2′ is the uncorrected absorbance value measured after addition of the modification composition. The optical path length is constant when the absorbance is measured in the same cuvette.

Depending on the orientation of the light source and detector, the change in volume may further cause a change in optical path length. Where there is a path length change, the value of AC2 may be calculated by the formula:

$$A C 2 = A C 2 ’ ( ( VCb ) / ( VCa ) ) * ( ( P Ca ) / ( PCb ) )$$

where VCa is the calibration sample volume prior to addition of the modification composition, VCb is the calibration sample volume after addition of the modification composition, PCa is the calibration sample optical path length prior to addition of the modification composition, PCb is the calibration sample optical path length after addition of the modification composition, and AC2′ is the uncorrected absorbance value measured after addition of the modification composition. The optical path length may vary before and after measurement when the absorbance is measured in a multi-well plate.

The calibration constant (Ct) may be calculated by the equation:

$$Ct = 1 / {( CCH )}^{*} ( AC 1 - AC 2 )$$

The units of concentration of hemoprotein (CCH) are preferably mg/L and the units of the calibration constant (Ct) are preferably L/(mg*Absorbance units).

It is appreciated that the calibration constant (Ct) may be determined from one calibration sample, or from a plurality of calibration samples. The plurality of calibration samples may contain at least two (e.g. 1, 2, 3, 4, 5, or more) calibration samples containing different concentrations of hemoprotein as mentioned elsewhere herein. The value of Ct may be calculated by calculating a fit of the change absorbance units to the change in concentration. In other words, the slope of the fitted line may be the value of Ct.

Typically the calibration constant (Ct) is determined for at least the spectrophotometer and the modification composition. It may further be determined for a reaction vessel type (e.g. cuvette, well of a multi-well plate), and/or optical path. Once determined, it does not need to be determined for each test sample or for each batch of test samples. It might need to be determined periodically (e.g. weekly, monthly) to account for instrument drift.

The concentration (CTH) of hemoprotein in the test sample may be determined by the equation:

$$CTH = 1 / {Ct}^{*} ( AT 1 - AT 2 )$$

The units of concentration of hemoprotein (CTH) is preferably mg/L and the units of the calibration constant (Ct) is preferably L/(mg*Absorbance units).

The values of AT1, AT2, AT2′, AC1, AC2, AC2′, and Ct are preferably scalar values. The scalar value is preferably obtained by measurement of absorbance values at a single discrete wavelength. Where absorbance values measured at a multiple different wavelengths, scalar values may be obtained from an average absorbance, a median absorbance, or an integrated absorbance.

It is an aspect the method includes a step of contacting the sample with the modification composition so that the porphyrin degrades. It is an aspect the method includes the step allowing the modification composition to react with the porphyrin so that the porphyrin degrades. It is an aspect the method includes the step degrading the porphyrin using the modification composition.

Provided is method for determining a concentration of a hemoprotein in a test sample, wherein the hemoprotein comprises heme prosthetic group containing porphyrin, the method comprising:
degrading the contacting the porphyrin using a modification composition;
measuring spectrophotometrically a change in an absorbance profile of the porphyrin caused by the degradation, wherein the absorbance profile is measured:
   at one wavelength within a spectrophotometric range of between 510 nm and 590 nm, or
   at a plurality of wavelengths at least one of which is within a spectrophotometric range of between 510 nm and 590 nm, and
      determining from the change in the absorbance profile, the concentration of hemoprotein in the sample.

Provided herein is a kit configured for carrying out the method described herein. Provided herein is a kit for determining a concentration of hemoprotein in a test sample, comprising a means for spectrophotometrically measuring modification of the hemoprotein in the sample, which means comprises a modification composition. The modification composition may contain 3% (v/v) hydrogen peroxide.

The means may comprise:
a stock solution of 30% (v/v) hydrogen peroxide,
a diluent for diluting the stock solution to a concentration of 1% to 5% (v/v).

The kit may further comprise one or more of:
a stock calibration standard (concentrated) for dilution containing hemoprotein at known concentration,
a plurality of calibration standards, each containing hemoprotein at a different known concentration,
a stock solution (concentrated) of diluent.

The diluent may be distilled water, physiological water. Diluents may be supplemented with stabilizing materials and microbial preservatives, as long as they do not interfere with the absorbance profile in the hemoprotein spectrum.

The kit may further comprise a spectrophotometer configured for spectrophotometric measurement within the heme absorbance spectrum range (e.g. between 510 nm and 590 nm, or preferably 570 nm to 580 nm, or more preferably 571 nm to 577 nm). The spectrophotometric measurement may be made at least within the heme absorbance spectrum range. The spectrophotometric measurement is preferably made only within the heme absorbance spectrum range.

Provided herein is a use of a modification composition for carrying out the method described herein.

Provided herein a computer-implemented method for determining a concentration of a hemoprotein in a test sample according to a method described herein.

Provided herein a computer-implemented method for determining a concentration of a hemoprotein in a test sample comprising:
receiving data of the spectrophotometrically measurements according to according to the method described herein, and
determining the concentration of hemoprotein according to according to the method described herein.

Provided herein a computer-implemented method for determining a concentration of a hemoprotein in a test sample comprising:
receiving data comprising a first absorbance profile of the test sample prior to contact with the modification composition;
receiving data comprising a second absorbance profile of the test sample after contact with the modification composition and after the hemoprotein has been modified;
determining from the change in the absorbance profile, the concentration of hemoprotein in the sample.

Provided herein a computer-implemented method for determining a concentration of a hemoprotein in a test sample comprising:
receiving data comprising a first absorbance profile (AT1) of the test sample prior to contact with the modification composition;
receiving data comprising a second absorbance profile (AT2) of the test sample after contact with the modification composition and after the hemoprotein has been modified;
receiving data comprising a calibration constant (Ct);
calculating the concentration (CTH) of hemoprotein by the equation:

$$CTH = {( 1 / ( Ct ) )}^{*} ( AT 1 - AT 2 ) ;$$

Further provided is a computing device or system configured for performing the computer-implemented method. The system typically comprises circuitry configured performing the method of the invention. Typically the circuitry comprises a processor and a memory.

Further provided is a computer program or computer program product having instructions which when executed by a computing device or system cause the computing device or system to perform the computer-implemented method.

Further provided is a computer readable medium having stored thereon the computer program or computer program product.

Further provided is a data stream which is representative of the computer program or computer program product.

### Example 1—Concentration Measurement by Present Method

Distilled water and physiologic water (0.9% (w/v) NaCl) were acquired from Baxter Healthcare SA (Zürich, Switzerland). Stabilised hydrogen peroxide (30% (v/v)) was obtained from Merck (Hohenbuch, Germany) and diluted with distilled water to a 3% (v/v) solution. A qualitative pool of hemoglobin was made through freeze-thawing a pool of expired citrated blood leftovers from routine analysis. An estimated concentration of the haemolysed supernatant was obtained through manual injection on the Sysmex 9000hematology analyser. A calibration sample with a final concentration of 55 mg/L was prepared from the haemolysed supernatant by dilution with demineralised water. A test sample of undiluted blood plasma prepared from whole blood of a subject.

### Calculation of Calibration Constant (Ct) According to the Present Method

To a 1.5 mL cuvette (1 cm fixed optical path length) was added 800 μL of the calibration sample at a concentration of 55 mg/L (CCH), and an absorbance (AC1) at 574 nm was measured. 400 μL of a 3% (v/v) hydrogen peroxide solution was added to the same cuvette, and mixed in. After 5 minutes, absorbance (AC2) at 574 nm was measured. Absorbance measurements were performed on an Atellica solutions Chemistry Analyser (Siemens) using an open channel. The calibration constant (Ct) was calculated using the equation:

$$Ct = 1 / {( CCH )}^{*} ( AC 1 - AC 2 ) = 1 / {( 332 )}^{*} ( 0.283 - 0.2385 ) = 0.000134$$

The value of AC2 was calculated as follows:

$$AC 2 = AC {2}^{′} ( ( VCb ) / ( VCa ) ) = ( 0.159 ) ( 1200 ) / ( 800 ) ) = 0.2385$$

### Determining Concentration Hemoprotein in Test Sample According to the Present Method

To a 1.5 mL cuvette (1 cm fixed optical path length) was added 800 μL of the test sample, wherein the test sample was prepared by dilution of the haemolysed supernatant-whose concentration was determined by a Sysmex 9000 hematology analyser-to have a concentration (CTH) of 55 mg/L hemoglobin. Absorbance (AS1) at 574 nm was measured. 400 μL of a 3% (v/v) hydrogen peroxide solution was added to the same cuvette, and mixed in. After 5 minutes, absorbance (AS2) at 574 nm was measured. Absorbance measurements were performed on an Atellica solutions Chemistry Analyser (Siemens) using an open channel. The hemoprotein concentration (CTH) was determined using the equation:

$$CTH = {( 1 / ( Ct ) )}^{*} ( AT 1 - AT 2 ) = {( 1 / 0.000134 )}^{*} ( 0.042 - 0. \text{.034} ) = 59.7 mg / L$$

The value of AT2 was calculated as follows:

$$AT 2 = AT {2}^{′} ( ( VTb ) / ( VTa ) ) = ( 0.0227 ) ( 1200 ) / ( 800 ) ) = 0.034$$

### Example 2—Correlation With Benzidine Method

Distilled water and physiologic water (0.9% (w/v) NaCl) were acquired from Baxter Healthcare SA (Zürich, Switzerland). Stabilised hydrogen peroxide (30% (v/v)) was obtained from Merck (Hohenbuch, Germany) and diluted with distilled water to a 3% (v/v) solution. A qualitative pool of hemoglobin was made through freeze-thawing a pool of expired citrated blood leftovers from routine analysis. An estimated concentration of the haemolysed supernatant was obtained through manual injection on the Sysmex 9000 hematology analyser. Calibration samples with a final concentration of 55 mg/L and 300 mg/L were prepared from this pool by dilution with demineralised water. A test sample was undiluted blood plasma prepared from whole blood of a subject.

A plurality of test samples having a concentrations ranging between 12 mg/L and 200 mg/L were prepared by selection of samples analysed in the routine laboratory using the benzidine method.

The concentration of hemoglobin in each test sample was measured by the present method using a calibration constant determined from the calibration sample, and also by the benzidine method.

The benzidine method is known in the art and contains a peroxidase step, in which haemoglobin catalyses the reaction of benzidine and hydrogen peroxide, generating a chromophore that absorbs at a wavelength of 600 nm. After a defined reaction period, the reaction mixture is quenched using excessive amounts of acetic acid.

The results (see FIG. 1) show a good correlation (correlation coefficient of 0.719, line (c)) between the benzidine method and present method for measurement of concentration of hemoglobin. The regression proved the slope and intercept not to be significantly different. Bland-altmann statistics proved limited number of significantly different results.

### Example 3—Correlation With Direct Spectrophotometric Measurement

The same test samples as used in Example 2 were also used an experiment to measure concentration of hemoglobin using a direct spectrophotometry method. The direct spectrophotometry method uses a spectrophotometric measurement technique containing the steps of dilution, followed by a measurement of the sample at different wavelengths, after which an algorithm provides a concentration value of haemoglobin. The algorithm was part of built-in program of a Roche Cobas 8000 spectrophotometer; no modification composition was used. The results show a poor correlation of measurements at the lower concentration range (5 to 40 mg/L) (see FIG. 2, arrow C) between the direct spectrophotometry method and present method for measurement of concentration of hemoglobin (see FIG. 2).

The values obtained when using the present method indicate the presence of hemoprotein at lower concentrations as confirmed by the Benzidine method, whereas the direct spectrophotometric method did not detect hemoprotein. The direct spectrophotometric method was not reliable at values <60 mg/L; considering 40 mg/L as a threshold for relevant haemolysis, the direct spectrophotometric method is not suitable for the detection of low-grade haemolysis.

### Example 4—Effect of Bilirubin

Several test samples containing bilirubin at a range of different concentrations were spiked with a known amount of hemoprotein (see FIG. 3). The concentration of hemoglobin according to the present method was measured. The recovery was calculated for each sample, which is an indication of an effect of bilirubin on the concentration measurement—a recovery of 100% is indicative of no effect of bilirubin on the measurement of concentration by the present method. The results (see FIG. 3) show an average recovery of 98±14%, indicative of a minimal effect of bilirubin.

### Example 5—Effect of Lipemia

A highly lipemic sample was serially diluted with non-lipemic plasma. All mixtures were spiked with a known amount of hemoprotein at a concentration of 100 mg/L (see FIG. 4). The concentration of hemoglobin according to the present method was measured. The recovery was calculated for each sample, which is an indication of an effect of lipid particles on the concentration measurement—a recovery of 100% is indicative of no effect of bilirubin on the measurement of concentration by the present method. The results (see FIG. 4) show an average recovery of 96±6%, indicative of a minimal effect of lipid particles as from an 8 fold serial dilution.

### Example 6—Screening of Different Candidate Modification Reagents

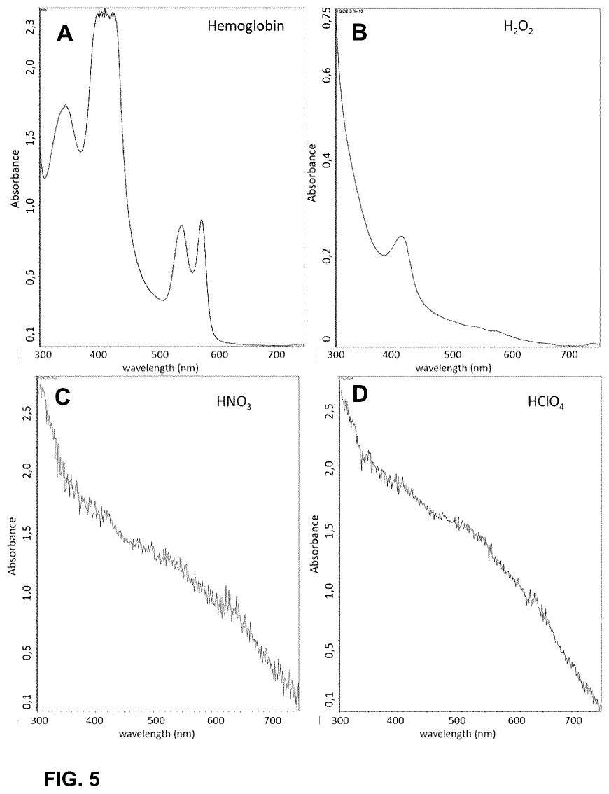

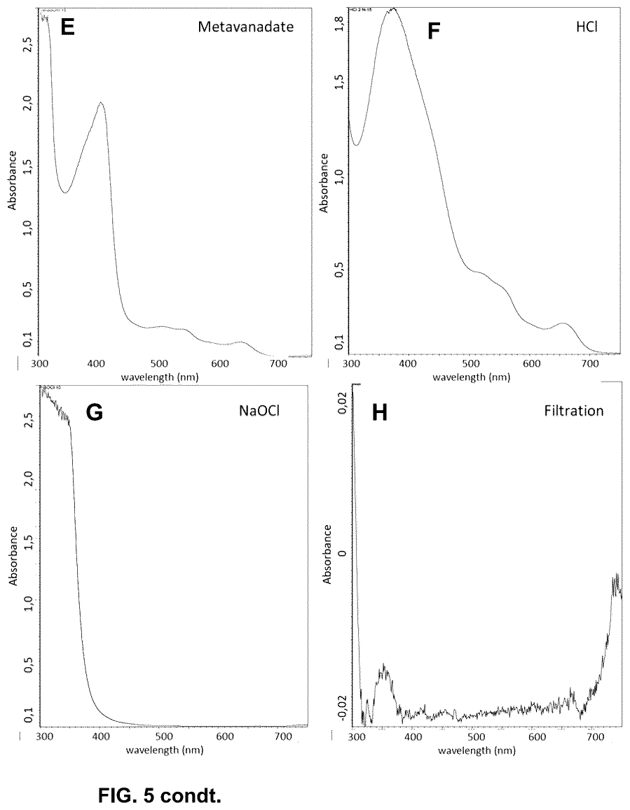

Citrated whole blood was hemolyzed using multiple freeze-thaw cycles. The supernatant was diluted in aqua dest to a final concentration of 1000 mg/L of free hemoglobin. 1 ml of this hemolysate was mixed with 500 μl of a modification composition containing the candidate modification reagent in Table 1 below. After 10-15 minutes, UV/VIS spectra were acquired between 300 and 750 nm. Results were compared qualitatively by comparison of spectrum in the heme-absorbing region of hemoglobin (no modification reagent) with hemoglobin (contacted with modification reagent). Spectra of candidate modification reagents useful in the present method are shown in FIGS. 5A to 5H.

TABLE 1

| Candidate | | | Quantitative |
|---|---|---|---|
| modification | Spectrum | | spectral changes |
| reagent | FIG. 5 | Appearance | in heme region? |
| None | A | Control - clear red | Control |
| H₂O₂ (3% v/v) | B | Pale red clear | Yes - intensity change |
| HNO₃ (2N) | C | Precipitation & | Yes - intensity change |
| | | turbidity - brown | |
| HClO₄ | D | Precipitation & | Yes - intensity change |
| (1% v/v) | | turbidity - brown | |
| Metavanadate | E | Pale brown clear | Yes - intensity change |
| (4 mmol/L) | | | and left shift |
| HCl (2N) | F | Brown clear | Yes - intensity change |
| | | | and left shift |
| NaOCl | G | Colourless solution | Yes - strong intensity |
| (5% v/v) | | | change |
| Filter | H | Removed all | Yes - strong intensity |
| | | components | change |
| NaOH (2N) | — | Green solution | Limited |
| MeOH | — | Clear red solution | None |
| | | appearance | |
| CAN | — | Pale brown clear | Limited |
| | | appearance | |
| Methanol | — | Severe precipitation | Not sufficient |
| and ZnSO₄ | | & turbidity - red | |

### Example 7—Wide Dynamic Range

Concentrations for free hemoglobin were measured using the presently-described method from blood samples of 750 patients in a clinical setting. The distribution of results is shown in FIG. 6. The results demonstrate that measurements can be made without dilution up to around 1000 mg/L. This compared with using benzidine where the range of measurements is estimated much lower (12.5-250 mg/L).

### Example 6—Free Hemoglobin Monitoring in ECMO Patient

Concentrations of free hemoglobin (fpHb) were monitored using the present method in a patient undergoing extracorporeal membrane oxygenation (ECMO) in a clinical setting. The results are shown in FIG. 7. The large arrows indicate a change to the ECMO circuit based on the reported fpHb in order to decrease hemolysis in the ECMO circuit. The low quantification limit allows the accurate follow-up and steadily increase of free haemoglobin prior to reaching levels that require an intervention. These trends, even in the low concentration zones, are of value to the clinician, because it is evidence of evolving in vivo hemolysis, rather than occasional in vitro hemolysis (which is an artefact). After an intervention, hemolysis was reduced, namely lower levels of fpHb were observed. The results demonstrate validity in a clinical setting. Advantageously, few steps are needed, there is less toxicity, smaller path lengths are needed, the measured are automatable, and continuously available.

1. A method for determining a concentration of a hemoprotein in a test sample, wherein the hemoprotein comprises heme prosthetic group containing porphyrin, the method comprising:
contacting the test sample with a modification composition comprising at least one modification reagent for degrading the porphyrin,
measuring spectrophotometrically a change in an absorbance profile of the porphyrin caused by the degradation, wherein the absorbance profile is measured:
at one wavelength within a spectrophotometric range of between 510 nm and 590 nm, or
at a plurality of wavelengths at least one of which is within a spectrophotometric range of between 510 nm and 590 nm, and
determining from the change in the absorbance profile, the concentration of hemoprotein in the sample.

2. The method according to claim 1, wherein the modification reagent is present in an amount to cause a loss of aromaticity to the porphyrin in the test sample.

3. The method according to claim 1, wherein the at least one modification reagent is an oxidation reagent, an acid, or a radical agent.

4. The method according to claim 1, wherein the at least one modification reagent is H₂O₂, HNO₃, HClO₄, metavanadate, HCl, NaOCl, sodium nitrate, tetrachloro-methane, bromo-trichloromethane, cumene hydroperoxide (CHP), t-butyl hydroperoxide, t-butyl perbenzoate, and dibenzoyl peroxide, nitrates, ozone, peroxides.

5. The method according to claim 1, wherein the change in absorbance profile caused by the modification composition is determined by:
measuring a first absorbance profile (AT1) of the test sample prior to contact with the modification composition;
measuring a second absorbance profile (AT2) of the test sample after contact with the modification composition and after the hemoprotein has been modified;
calculating a difference between the first (AT1) and second (AT2) absorbance profiles.

6. The method according to claim 1, wherein the step of
determining from the change in absorbance profile the concentration of hemoprotein in the test sample
comprises applying a calibration constant (Ct) to the change in absorbance profile.

7. The method according to claim 6 wherein the concentration (CTH) of hemoprotein in the test sample is determined by the equation:
$$CTH = ( 1 / ( Ct ) ) * ( AT 1 - AT 2 )$$
wherein AT2 has been corrected for any change in volume caused by contacting the test sample with the modification composition.

8. The method according to claim 7, wherein the calibration constant (Ct) is determined by measuring spectrophotometrically a change in absorbance profile caused by modification of a known concentration (CCH) of hemoprotein in a calibration sample.

9. The method according to claim 8, wherein
the change in absorbance profile caused by modification of the known concentration (CCH) hemoprotein in the calibration sample is determined by:
measuring a first absorbance profile (AC1) of the calibration sample prior to addition of the modification composition;
measuring a second absorbance profile (AC2) of the calibration sample after addition of the modification composition and after the hemoprotein has been modified; and
calculating a difference between the first absorbance profile (AC1) and the second absorbance profile (AC2), and
the calibration constant (Ct) is calculated by the equation:
Ct=1/(CCH)*(AC1−AC2) wherein AC2 has been corrected for any change in volume caused by contacting the calibration sample with the modification composition.

10. The method according to claim 9, wherein the concentration (CTH) of hemoprotein in the test sample is determined by the equation:
$$CTH = 1 / Ct * ( AT 1 - AT 2 )$$
wherein AT2 has been corrected for any change in volume caused by contacting the test sample with the modification composition.

11. The method according to claim 8, the change in an absorbance profile of the porphyrin caused by the degradation, comprises:
a reduction in absorbance at one wavelength within a spectrophotometric range of between 510 nm and 590 nm, or
a reduction in absorbance at a plurality of wavelengths at least one of which is within a spectrophotometric range of between 510 nm and 590 nm.

12. The method according to claim 1, wherein the hemoprotein is hemoglobin.

13. The method according to claim 1, wherein the hemoprotein in the test sample is present in blood plasma, urine or cerebrospinal fluid.

14. A kit for determining a concentration of hemoprotein in a test sample according to a method of claim 1, comprising a means for spectrophotometrically measuring modification of the hemoprotein in the sample, which means comprises a modification composition.

15. The kit according to claim 14, wherein the means comprises
a stock solution of 30% (v/v) hydrogen peroxide,
a diluent for diluting the stock solution to a concentration of 1% to 5% (v/v).

16. The kit according to claim 14, further comprising:
a stock calibration standard for dilution containing haemoprotein at known concentration, and/or
a plurality of calibration standards, each containing hemoprotein at a different known concentration.

17. A computer-implemented method for determining a concentration of a hemoprotein in a test sample according to claim 1.

18. A computer-implemented method for determining a concentration of a hemoprotein in a test sample comprising
receiving data of the spectrophotometrically measurements according to claim 1, and
determining the concentration of hemoprotein according to claim 1.
